(19) United States
(12) Patent Application Publication
(10) Pub. No.: US 2015/0217103 A1
(43) Pub. Date: Aug. 6, 2015

(54) Arcuate Guide for Intravenous Tubes

(71) Applicant: Henry P. Serafini, Leominster, MA (US)

(72) Inventor: Henry P. Serafini, Leominster, MA (US)

(21) Appl. No.: 14/460,865

(22) Filed: Aug. 15, 2014

Related U.S. Application Data
(60) Provisional application No. 61/936,716, filed on Feb. 6, 2014.

(51) Int. Cl. A61M 39/08

(52) U.S. Cl. CPC A61M 39/08; A61M 2039/0009

(57) ABSTRACT

An arcuate guide for retaining and guiding an intravenous tube over an angular change of direction. The arcuate guide has an arcuate retention structure with an arcuate channel structure and a plurality of locking members. The arcuate channel structure has an arcuate channel therein for receiving and guiding the intravenous tube over an angular change of direction, and the plurality of locking members are disposed in substantial alignment with the arcuate channel. The arcuate channel can be substantially U-shaped, such as by having a rounded base portion and arcuate first and second legs. The channel can have radiused distal edges. The arcuate channel can be adjustable in angular configuration to permit an adjustment of the angular change of direction of the intravenous tube. The locking members can have opposed legs spaced closer than opposed sidewalls of the arcuate channel. The opposed legs can be resiliently deflectable.

### PRIORITY

This application claims priority to U.S. Provisional Patent Application No. 61/936,716, filed Feb. 6, 2014, which is incorporated herein by reference.

### FIELD OF THE INVENTION

The present invention relates generally to the medical field. More particularly, disclosed and protected herein is an arcuate guide for intravenous tubes capable of guiding intravenous tubes over a smooth, arcuate angle of directional change to promote and maintain unobstructed flow and administration of liquids and liquid medicines to patients intravenously.

### BACKGROUND OF THE INVENTION

It will be appreciated that the effective and reliable provision of liquids and medicines to patients is critical to proper medical treatment. Accordingly, the prior art has disclosed numerous systems and methods for delivering liquid medicines and liquids to patients. Among the most fundamental methods for providing liquids and liquid medicines to patients is intravenous tubing. When functioning properly, IV tubing is desirable as a delivery method for its ability to provide controlled flow of liquid hydration, nutrients, and medicine in an intermittent or continuous manner without requiring constant human attention.

In a typical intravenous delivery method, a volume of liquid is retained in a liquid supply vessel, such as a polymeric bag. The liquid may be chosen simply for hydration, or the liquid may be essentially medicinal. Additionally or alternatively, medicines in liquid form may be retained and supplied in combination with hydration liquids. A liquid supply conduit, commonly referred to as an IV tube, has a first end fluidically connected to the liquid supply vessel and a second end fluidically connected to the patient, typically by a hollow intravenous (IV) needle. With the liquid supply vessel connected to the patient by the IV tube, liquid can be permitted to flow to the patient. Liquid flow can be selectively controlled, such as to be provided intermittently or, additionally or alternatively, at a desired flow rate. Liquid can flow automatically, such as under the force of gravity. Alternatively, liquid can be caused to flow at a given rate or based on time or on demand by a pumping mechanism, such as a peristaltic pump.

In practice, the liquid supply vessel is commonly located at the head of or behind the patient's bed. With that, the IV tube must pass from the supply vessel, along the bed, and to the patient's body. The IV needle, which is disposed at the distal end of the IV tube, is inserted into a vein of the patient to permit the flow of liquids. It is common for surgical tape to be used to secure the IV needle and one or more portions of the flexible IV tube to the patient's body, such as the patient's hand or arm.

Based on the relative locations and orientations of the liquid supply vessel, the IV tube and needle, and the patient's arm or other body part, it is often necessary for the distal portion of the IV tub to be required to undergo a substantial change in direction. The change in direction of the IV tube necessary to become aligned or at least substantially aligned with the IV needle will vary, but it can be 180 degrees or more. Unfortunately, such extreme directional changes can cause the IV tubing to pinch or otherwise become occluded. Indeed, even normal movement of the patient can cause the IV tube to become pinched or blocked adjacent to the distal end of the tube. When the IV tube is pinched, the flow of liquids being administered can be limited or even entirely blocked. In either case, the patient can be deprived of needed liquids and medicines thereby leading to unnecessary patient discomfort, trauma, and lack of necessary liquids and nutrients. Moreover, occlusion of the IV tube can lead to blood clots at the insertion site and increases in the risk of infection and other complications for the patient.

When a monitored IV tube becomes blocked or otherwise malfunctions, visual alarm signals, such as flashing or other visual indications, are triggered, and disruptive beeps, buzzers, or other audible alarms sound. These arm signals are designed to draw the attention of medical staff to the malfunction and normally require intervention by the medical staff to correct the malfunction. The visual and audible alarms and the need for staff intervention to eliminate the pinching of the IV tube are inconvenient and disruptive to the patient, particularly during rest, and they represent an inefficient use of the responding staff's time. Where multiple alarms sound on a continuing basis, alarm fatigue, where staff begins to pay less and perhaps no attention to alarms, can begin to set in. As a result, alarm response time can become dangerously long. It is thus apparent to the present inventor that minimizing unnecessary alarms would be highly desirable.

Under current practices, medical staff commonly attempt to secure the IV tube to the patient's arm with the required directional change between the liquid supply vessel and the patient's body by the use of simple medical tape. However, creating a stable and continuous curve is often problematic, and securing the IV tube can be unreliable. Patient movement and other factors can cause the tape to shift, loosen, or become entirely separated, allowing the IV tube to be pulled or pinched and become occluded. With that, maintaining a curved transition without undesirable kinking is often not possible so that patients and staff must experience the malfunctions, alarms, and other deleterious effects summarized above.

With a knowledge of the foregoing, the present inventor has recognized that it would be advantageous to provide a guide for retaining IV tubes in a secure and smoothly curved formation relative to a patient's body thereby to prevent the tube from becoming pinched and blocked and to avoid the alarms, discomfort, dangers, and inconvenience associated therewith.

### SUMMARY OF THE INVENTION

The present invention is thus founded on the basic object of providing a guide for retaining intravenous and other tubes in relation to a body, such as a human body, and for stably securing such tubes to the body in a smooth, arcuate configuration.

In certain embodiments of the arcuate guide for intravenous tubes, an object is to guide intravenous tubes over a smooth turn of approximately 180 degrees.

Another object of the particular embodiments of the invention is to provide an arcuate guide for intravenous tubes wherein the angle of directional change over which the intravenous tube is guided can be adjusted to accommodate, among other things, different relative locations of the patient and the liquid supply vessel, different tubes, and other circumstances.

A related object of embodiments of the arcuate guide for intravenous tubes is to guide intravenous tubes over an arcuate turn from a patient's body to a liquid supply vessel.

A further object of the invention is to provide an arcuate guide for intravenous tubes that prevents the tube from becoming excessively bent or kinked and, in so doing, avoids limitations to the flow of liquids through the tube by bends and kinks.

An underlying object of the invention is to provide an arcuate guide for intravenous tubes that reduces the need for human intervention to permit proper liquid flow and that minimizes the alarm conditions and instances deriving from blocked liquid flow due to excessive bending and kinking.

A more particular object of embodiments of the invention is to provide an arcuate guide for intravenous tubes that is capable of flexing to accommodate the contour of the patient's body and the movement of the patient.

A related object of embodiments of the invention is to provide an arcuate guide for intravenous tubes that is capable of remaining fixed in place against inadvertent displacement or removal thereby to prevent, among other things, unintended movement or removal of an intravenous or other tube and the concomitant discomfort and inconvenience attended thereto.

Still another object of embodiments of the invention is to provide an arcuate guide for intravenous tubes that avoids blood clots and other complications deriving from pinching, occlusion, or inadvertent removal of the tube.

These and further objects and advantages of the present invention will become obvious not only to one who reviews the present specification and drawings but also to those who have an opportunity to experience an embodiment of the arcuate guide for intravenous tubes disclosed herein in use. However, it will be appreciated that, although the accomplishment of each of the foregoing objects in a single embodiment of the invention may be possible and indeed preferred, not all embodiments will seek or need to accomplish each and every potential advantage and function. Nonetheless, all such embodiments should be considered within the scope of the present invention.

In carrying forth these objects, one embodiment of the arcuate guide for retaining and guiding an intravenous tube over an angular change of direction can be considered to be founded on an arcuate retention structure with an arcuate channel structure and a plurality of locking members. The arcuate channel structure has an arcuate channel therein for receiving the intravenous tube and guiding the intravenous tube over an angular change of direction, and the plurality of locking members are retained in substantial alignment with the arcuate channel in the arcuate channel structure.

While other shapes are possible within the scope of the invention, the arcuate channel in the arcuate channel structure could be substantially U-shaped. Moreover, the arcuate channel in the arcuate channel structure can have a generally U-shaped cross section for receiving the intravenous tube, and the arcuate channel can have radiused distal edges to permit the convenient insertion of the tube therein.

Embodiments of the arcuate guide are contemplated wherein the arcuate channel in the arcuate channel structure can be selectively adjustable in angular configuration. With that, the angular change of direction of the intravenous tube can be selectively adjusted. By way of example, the arcuate channel structure can have first and second legs with the arcuate channel in the arcuate channel structure having portions retained by the first and second legs of the arcuate channel structure. The relative orientation of the first and second legs can be selectively adjustable to adjust the angular change of direction of the intravenous tube.

In certain practices of the invention, one or more hinge portions could be disposed along the arcuate channel structure to contribute to the selective adjustment in angular configuration of the arcuate channel structure. In such practices of the invention, there could be at least first and second central locking members disposed at a mid-portion of the arcuate channel structure, and the hinge portion can be disposed between the first and second central locking members that are disposed at the mid-portion of the arcuate channel structure.

The arcuate guide can further include a support member, and the first and second legs can be selectively retained by the support member at selected angles of orientation. The support member could, for example, comprise a cross member or a base panel. In either construction, a plurality of apertures could be spaced across the support member, and the first and second legs can be selectively retained relative to the plurality of apertures. For example, the first and second legs could be selectively retained relative to the plurality of apertures by teeth that project from the first and second legs to be selectively received into one or more of the plurality of apertures.

Where the arcuate channel in the arcuate channel structure is selectively adjustable in angular configuration, it can permit an angular change of direction over a given range of angular change of direction. By way of example and not limitation, the range of angular change of direction could encompasses from less than 180 degrees to greater than 180 degrees.

The arcuate channel in the arcuate channel structure can be defined by first and second opposed sidewalls separated by a given effective distance. Each locking member can have opposed legs separated by a given effective distance. The effective distance between the opposed legs of the locking members can be less than the effective distance between the first and second opposed sidewalls of the arcuate channel in the arcuate channel structure.

A locking member disposed adjacent to a first end of the arcuate channel in the arcuate channel structure, and a locking member can be disposed adjacent to a second end of the arcuate channel in the arcuate channel structure. The opposed legs of the locking members could formed from substantially rigid material, including but not limited to plastic or metal, and the opposed legs of the locking member can be resiliently deflectable.

A support member can be provided for retaining the arcuate retention structure. The support member can have a first face and a second face. Adhesive can be disposed on at least the first face of the support member for selectively retaining the support member and the arcuate guide relative to a patient's skin. Embodiments are further contemplated wherein adhesive is disposed on the second face of the support member for retaining the arcuate retention structure. In any event, the support member can be a panel of material for being interposed between the arcuate retention structure and a patient's skin, and a window can be disposed in the support member for permitting a visual perception of the patient's skin.

One will appreciate that the foregoing discussion broadly outlines the more important goals and features of the invention to enable a better understanding of the detailed description that follows and to instill a better appreciation of the inventor's contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF DRAWINGS

In the accompanying drawing figures:

FIG. 1 is a top plan view of an arcuate guide for intravenous tubes according to the invention;

FIG. 2 is a top plan view of the arcuate guide for intravenous tubes of FIG. 1 in a partially disassembled configuration;

FIG. 3A is a top plan view of the arcuate guide for intravenous tubes of FIG. 1 in a further disassembled configuration;

FIG. 3B is a cross section of the arcuate channel structure taken along the line 3B-3B in FIG. 3A;

FIG. 4 is a view in front elevation of the arcuate guide for intravenous tubes of FIG. 1 in a disassembled configuration;

FIG. 5 is a top plan view of the arcuate guide for intravenous tubes of FIG. 1 in position relative to a patient's body retaining an IV tube;

FIG. 6 is a top plan view of an alternative arcuate guide for intravenous tubes pursuant to the invention; and

FIG. 7 is a top plan view of the arcuate guide for intravenous tubes of FIG. 6 in position relative to a patient's body retaining an IV tube.

### DETAILED DESCRIPTION OF PREFERRED EMBODIMENTS

The arcuate guide for intravenous tubes disclosed herein is subject to a wide variety of embodiments. However, to ensure that one skilled in the art will be able to understand and, in appropriate cases, practice the present invention, certain preferred embodiments of the broader invention revealed herein are described below and shown in the accompanying drawing figures.

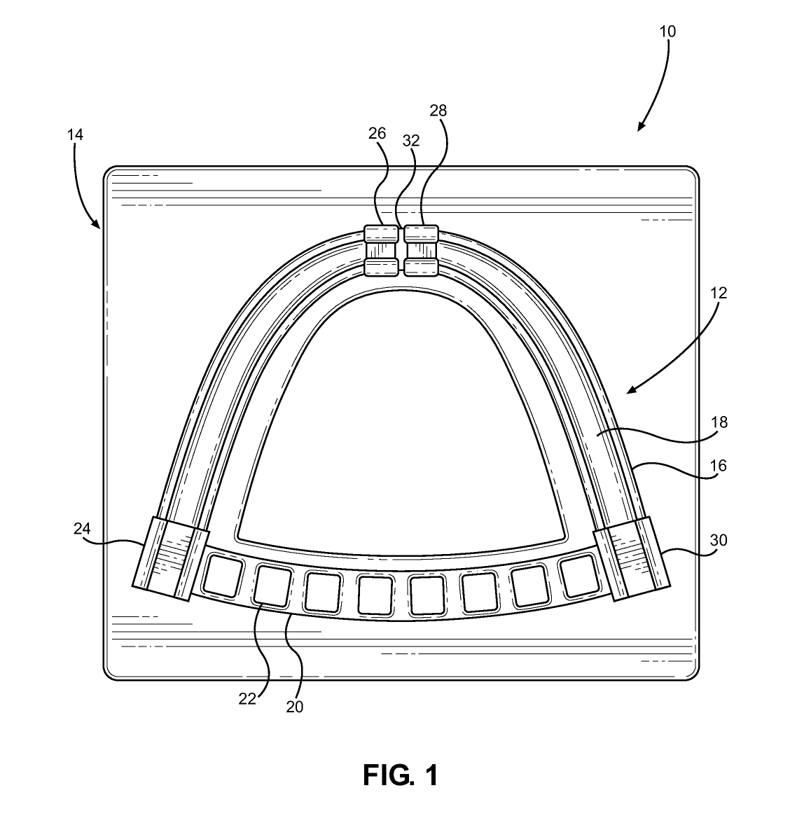

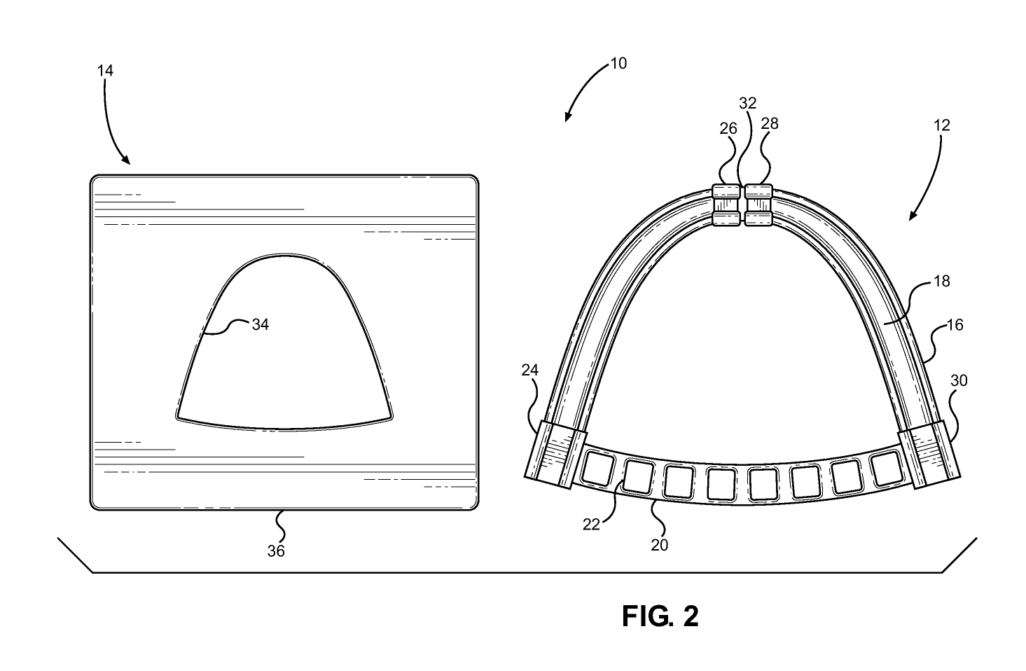

Turning more particularly to the drawings, an arcuate guide for intravenous tubes according to the present invention is indicated generally at 10 in FIGS. 1 through 5. The arcuate guide for intravenous tubes 10 can be considered to be founded on an arcuate retention structure 12 with a substantially U-shaped, arcuate channel structure 16. The arcuate channel structure 16 can be formed with a single, continuous channel section. Alternatively, the arcuate channel structure 16 can be formed with multiple channel sections. The invention should not be interpreted as being limited to either construction except as it might be expressly limited by the claims.

It will be understood that reference to a U-shaped structure should not be interpreted to limit the invention to a U-shape of any particular geometry. By way of example and not limitation, the arcuate channel structure 16 need not have parallel or even near-parallel legs. As is illustrated, for instance, the arcuate channel structure 16 can have a smooth bottom portion and first and second legs that project therefrom at generally equal, outward angles. The arcuate channel structure 16 could just was well be characterized merely as being arched shaped or by some other description of its arcuate form. Indeed, it is contemplated within the scope of the invention that arcuate channel structures 16 could have different shapes, such as J-shapes, S-shapes, or some other smooth, arcuate shape, each within the scope of the inventor's protection except as the invention might be expressly limited.

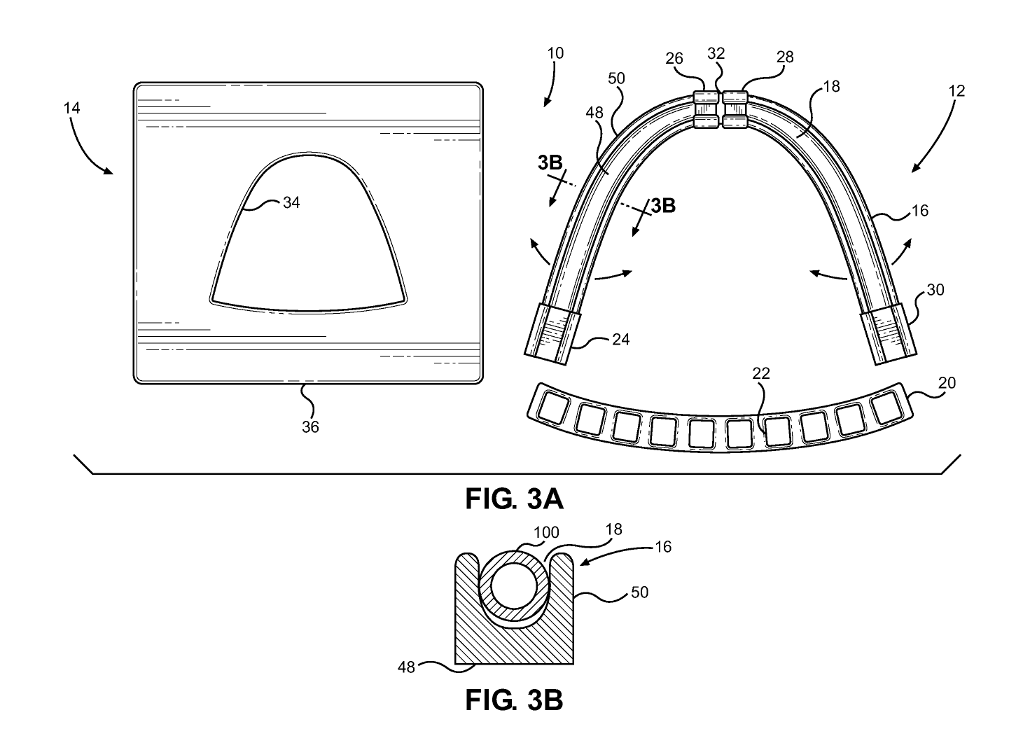

As best shown perhaps in FIG. 3B, the arcuate channel structure 16 can have a base portion 48 and first and second opposed sidewalls 50. A channel 18 with a U-shaped cross section is established with the base portion 48 as the bottom thereof and the sidewalls 50 defining the sides of the channel 18. The distal ends of the sidewalls 50 can be smoothly radiused at their inner edges and, potentially, their outer edges. The base portion 48 in this manifestation of the invention has a flat base surface. In certain practices of the invention, the arcuate channel structure 16 can be formed form a relatively soft and pliable material, such as but not necessarily limited to rubber or plastic foam, rubber, semi-soft plastic material, or any other suitable material or combination thereof. The chosen material or materials can be substantially or entirely latex free.

Under this construction, intravenous tubes 100 within a given range of diameters can be received into the U-shaped channel 18 thereby to be guided to pursue the arcuate path of the arcuate channel structure 16. Intravenous tubes 100 and the U-shaped channel 18 could be relatively sized to have tubes 100 with diameters less than the distance between the opposed sidewalls 50 so that the tubes 100 can be loosely received, or tubes 100 can have diameters equal to or marginally greater than the distance between the opposed sidewalls 50 so that the tubes 100 can be frictionally engaged and retained by the sidewalls 50.

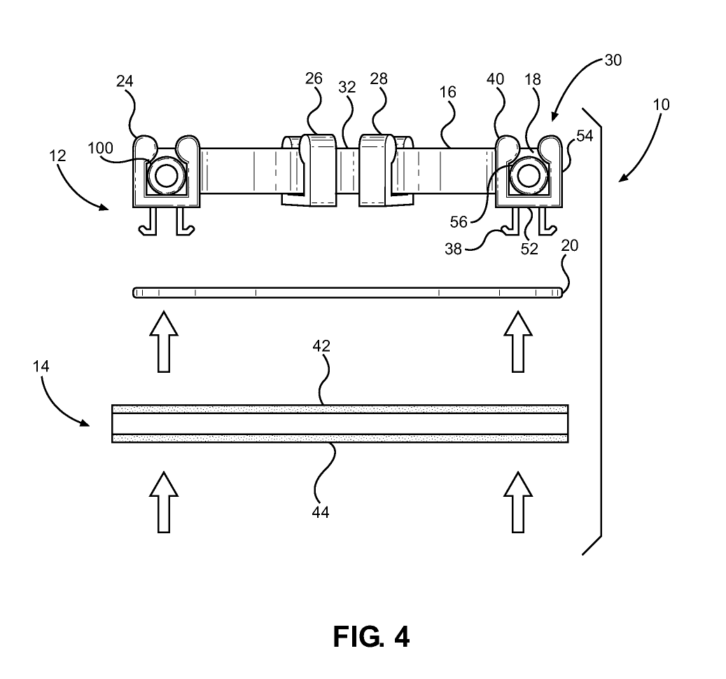

A plurality of tube locking members 24, 26, 28, and 30 are disposed along, contiguous with, or substantially contiguous with the U-shaped channel 18 of the arcuate channel structure 16 in alignment with the channel structure 16. In the depicted embodiment, there are four locking members 24, 26, 28, and 30, but it will be understood that fewer or more locking members 24, 26, 28, and 30 could be provided. As can be perceived by reference to FIG. 4, each of the locking members 24, 26, 28, and 30 can have a base portion 52 and first and second opposed legs 54 with inwardly disposed protuberances or portions 40, which can be bulbous protuberances 40. With that, a retention volume 56 is formed between the opposed legs 54. The retention volume 56 has a narrowed entrance as established by the bulbous protuberances 40. The locking members 24, 26, 28, and 30 can be formed from a substantially rigid material, such as a plastic or metal. The opposed legs 54 can be fixed or resiliently deflectable.

Under this construction, an intravenous tube 100 with an outer diameter within a given range greater than the distance between the bulbous portions 40 can be pressed or snapped between the bulbous portions 40 and into the retention volume 56. With that, the inwardly disposed portions 40 of the opposed legs 54 of each of the locking members 24, 26, 28, and 30, and perhaps the opposed legs 54 themselves if the distance between them is less than the diameter of the tube 100, will tend to retain the intravenous tube 100 against inadvertent displacement from the retention volume 56 and the arcuate channel structure 16 in general.

Indeed, it is possible and within the scope of the invention, except as it might be expressly limited in the claims, to have locking members 24, 26, 28, and 30 with opposed legs 54 that do not have inwardly disposed protuberances but instead merely have opposed legs 54 with distances between at least portions thereof less than the anticipated outer diameter of the tube 100. The opposed legs 54 could be smooth, textured, or with gripping formations therealong such as teeth, ridges, angled fins, barbs, or any other gripping formation. The legs 54 could be substantially parallel, or they could be angled, such as by being angled toward one another whereby the distance between them will narrow toward their ends. The distance between the opposed legs 54 at their narrowest portions, whether at the inwardly disposed portions 40, any gripping formations, the tips of the legs 54, or even the overall legs 54 themselves, can be narrower than the outer diameter of the intended tube 100 and narrower than the distance between the first and second opposed sidewalls 50 of the arcuate channel structure 16. With that, the legs 54 of the locking members 24, 26, 28, and 30 will exert a locking force on the tube 100.

With it again being noted that fewer or more locking members 24, 26, 28, and 30 could be provided, the four locking members 24, 26, 28, and 30 in the depicted embodiment are disposed with a locking member 24 disposed at a distal end of the first leg of the U-shaped arcuate channel structure 16 and a locking member 30 disposed at a distal end of the second leg of the arcuate channel structure 16. Central locking members 26 and 28 are disposed at a mid-portion of the arcuate channel structure 16, such as at what could be considered the base of the U-shape. Here, there are first and second central locking members 26 and 28, but it would be possible to have just one central locking member 26 or 28 or more than two central locking members 26 and 28 that could be disposed in juxtaposition or spaced along the structure 16. The central locking members 26 and 28 could be separate, or they could be connected, such as by being integrally formed or in some other manner.

With the locking members 24, 26, 28, and 30 so disposed, a segment of an intravenous tube 100 can be securely retained, such as by being snapped into place to be retained by the locking members 24, 26, 28, and 30, to undergo a generally U-shaped directional change. The locking members 24 and 30 will retain first and second ends of the segment of the tube 100, the central locking members 26 and 28 will retain mid-portions of the segment of the tube 100, and portions of the tube 100 between the locking members 24, 26, 28, and 30 will be retained in the U-shaped channel 18 of the arcuate channel structure 16.

The arcuate structure formed by the arcuate channel structure 16 and the locking members 24, 26, 28, and 30 can be stabilized in a given configuration by, for example, one or more backing or cross members 20. Here, the cross member 20 comprises an arcuate band of material, which could be a rigid, substantially rigid, or flexible material. It will be understood that the cross member 20 could be differently formed, such as in the form of a larger panel or some other structure. In one non-limiting instance, the cross member 20 could be formed from a plastic. The arcuate channel structure 16 can be fixedly or selectively coupled to the cross member 20.

In certain practices of the invention, the legs of the arcuate channel structure 16 can be adjustably coupled to the cross member 20. With that, the relative orientations of the legs of the arcuate channel structure 16 can be adjustable, and the angle of directional change of a retained intravenous tube 100 can be adjustable within a given range. For instance, where the legs of the arcuate channel structure 16 are coupled at or adjacent to the outboard ends of the cross member 20, the legs of the arcuate channel structure 16 will project somewhat away from one another and the angle of directional change of the tube 100 may be at or near a minimum, such as some angle less than 180 degrees. However, where the legs of the arcuate channel structure 16 are coupled toward a mid-portion of the cross member 20, the legs of the arcuate channel structure 16 will project somewhat toward one another and the angle of directional change of the tube 100 will be greater, such as at some angle in excess of 180 degrees. Of course, the range of adjustability of the angle of directional change will depend on the lateral dimension of the cross member 20.

The adjustability of the angular configuration of the arcuate channel structure 16 can, by way of example and not limitation, be permitted by a flexibility in the arcuate channel structure 16, through the coupling of the arcuate channel structure 16 with the locking members 24, 26, 28, and 30, or by some combination thereof or some other method. In certain embodiments, a hinge 32 could be formed, such as through a living hinge, through inherent flexibility, or some other method, to contribute to the relative adjustability of the legs of the arcuate channel structure 16.

The flexibility of the arcuate channel structure 16, potentially with the hinge 32, can also permit its conformance to the contour of a patient's body while permitting movement of the patient and avoiding discomfort and dislodging of the arcuate guide for intravenous tubes 10. In this respect, the hinge 32 could be a bi-directional hinge to permit not only lateral movement of the legs of the arcuate channel structure 16 but also relative pivoting between the legs about a longitudinal axis of the arcuate channel structure 16.

The legs of the arcuate channel structure 16 could be retained relative to the cross member 20 in any effective manner, including adhesive, mechanical engagement, fasteners, or any other fastening or retention mechanism. In this example of the invention, the cross member 20 has a plurality of apertures 22 spaced therealong, and each leg has a tooth 38 or opposed teeth 38, possibly with lateral locking projections, that can be selectively engaged with an aperture 22 or apertures 22, such as in a snap-fit engagement. Under this construction, the arcuate channel structure 16 and the cross member 20 can be readily and selectively engaged, and the angle of directional change provided by the arcuate guide for intravenous tubes 10 can be selectively modified.

The arcuate retention structure 12, such as might be formed with the coupling of the arcuate channel structure 16 and the cross member 20, can be retained relative to a patient's body by any effective mechanism. In one possible embodiment, as seen for example in FIG. 4, a base panel 14 can have a first adhesive surface 42 for engaging and retaining the coupled arcuate channel structure 16 and the cross member 20 and a second, opposite adhesive surface 44 for engaging and adhering to a patient's skin. As FIG. 4 shows, it is possible for the base panel 14 to comprise a rectangular panel of foamed plastic, plastic, metal, rubber, or any other material with adhesive coatings on the faces thereof to form the adhesive surfaces 42 and 44. The base panel 14 can be pre-cut. Additionally or alternatively, it could be customized in anticipation of a given application. The surfaces 42 and 44 of the base panel 14 could have non-adherent cover sheets (not shown) that can be removed to permit adherence to the arcuate retention structure 12 and the patient's skin.

It will be understood, however, that the arcuate guide for intravenous tubes 10 is not limited to having a base panel 14 and could be retained relative to a patient's body in some other manner, such as by tape, adhesive, or some other method. Conversely, it should be understood that the adhesive base panel 14 could in theory act as a cross member permitting a selective coupling and retention of the legs of the arcuate channel structure 16. In such embodiments, a dedicated cross member 20 might be foregone.

In any case, the base panel 14 in this manifestation of the invention comprises a rectangular panel 36 with radiused corners. The base panel 14 has a window 34 disposed in a central portion thereof. The window 34 could be a translucent or transparent portion, or it may be entirely open, as by a cutout portion. Here, the window 34 is defined by an arcuate upper edge generally matching the shape of the arcuate channel structure 16 and a generally straight or somewhat curved lower edge. The window 34 can match the general shape of the inner edges of the arcuate channel structure 16 and the cross member 20 while being slightly smaller and inboard thereof. With such a window 34 provided, a visual inspection of the localized area of skin of the patient is permitted, which can be assistive, for example, in avoiding malfunctions and perceiving damage to the site.

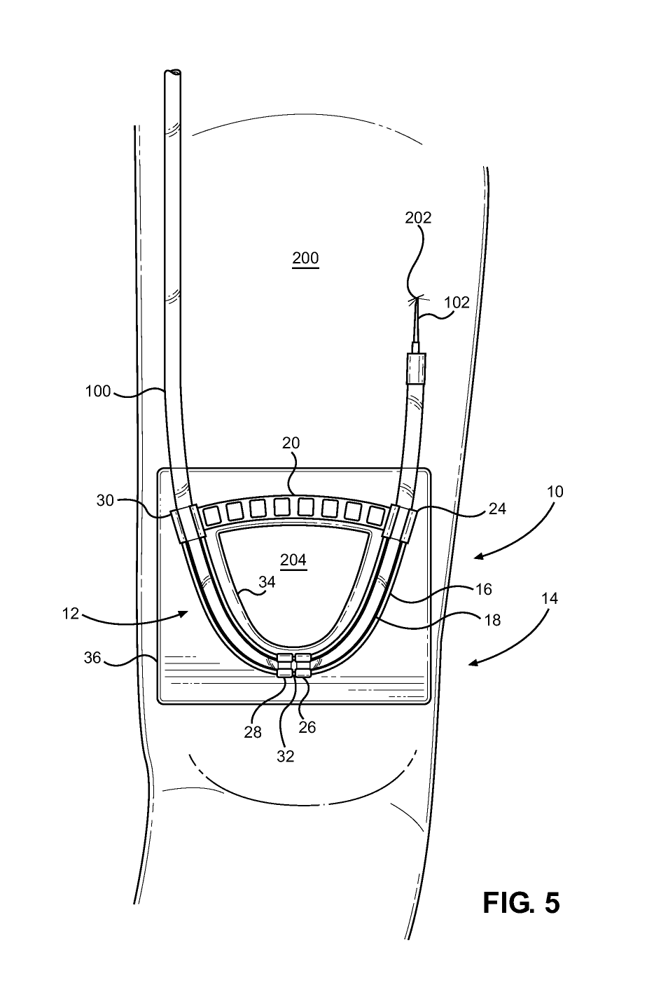

As shown and described, an arcuate guide for intravenous tubes 10 as taught herein can be applied to a patient's body 200 to guide an intravenous tube 100 smoothly and in an arcuate manner of over an angle of directional change as suggested by FIG. 5. While the steps and the order of steps can vary according to the invention, one step might involve adjusting the arcuate channel structure 16 to cause there to be a given angle between the legs of the structure 16. The cross member 20 and the arcuate channel structure 16 can be coupled to secure the arcuate channel structure 16 at the desired angle. The joined arcuate channel structure 16 and the cross member 20 can be secured to the adhesive panel 14, and the base panel 14 can be secured to a patient's body 200, such as the patient's arm. A portion of skin 204 aligned with the window 34 can be perceived. An intravenous tube 100 can be snapped into place within the locking members 24, 26, 28, and 30 to be retained by the arcuate channel structure 16 to undergo a given angle of directional change over a smooth, arcuate curvature. The intravenous needle 102 at the distal end of the intravenous tube 100 can be inserted into an injection site 202 on the patient's body 200. With this, liquid medicines and hydrative and nutritive liquids can be provided to the patient with the intravenous tube 100 smoothly changing direction, such as between a liquid supply vessel and the patient's arm 200, with reduced risks of kinking, occlusion, discomfort, and unintended withdrawal.

So disclosed, the arcuate guide for intravenous tubes 10 could be provided in different sizes and configurations. By way of example, the arcuate guide for intravenous tubes could have different sizes of some or all components to be purposed for adults, children, and infants. Moreover, the arcuate guide for intravenous tubes 10 could have various shapes and sizes to accommodate different parts of the body, such as the hand, the arm, the leg, or some other IV insertion site. The arcuate channel structure 16, the locking members 24, 26, 28, and 30, and other components can be geometrically shaped and sized to accommodate differently-sized tubing. Where necessary or desirable, the arcuate guide for intravenous tubes 10 could have indicia (not shown) thereon, such as of acceptable sizes of tubes 100, to ensure successful tube insertion and effective tube retention.

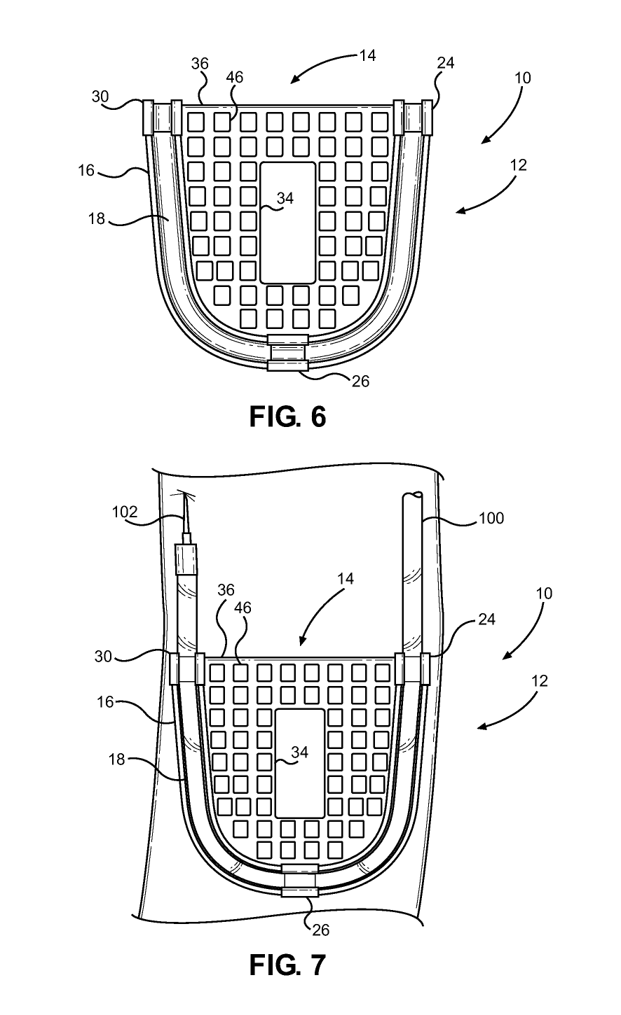

The present invention for an arcuate guide for intravenous tubes 10 is broader than any single embodiment. Arcuate guides for intravenous tubes 10 can readily pursue different constructions within the scope of the invention beyond the example shown and described above. By way of further example and not limitation, another embodiment of an arcuate guide for intravenous tubes 10 according to the invention is again indicated at 10 in FIGS. 6 and 7. There, the arcuate guide for intravenous tubes 10 can again be considered to be founded on an arcuate retention structure 12 with a substantially U-shaped, arcuate channel structure 16. As before, the arcuate channel structure 16 can be formed with a single, continuous channel section or with multiple channel sections. The arcuate channel structure 16 can, as in the earlier embodiment, have a channel 18 with a U-shaped cross section established therein. Intravenous tubes 100 within a given range of diameters can be received into the U-shaped channel 18 thereby to be guided to pursue the arcuate path of the arcuate channel structure 16.

A plurality of tube locking members 24, 26, and 30, in this case comprising three locking members 24, 26, and 30, are disposed along, contiguous, or substantially contiguous with the U-shaped channel 18 of the arcuate channel structure 16 in alignment therewith. As in the previous embodiment and as is shown in FIG. 4, each of the locking members 24, 26, and 30 can have a base portion 52 and first and second opposed legs 54 with inwardly disposed protuberances or portions 40, which can be bulbous protuberances 40. A retention volume 56 is thus formed between the opposed legs 54 with a narrowed entrance as established by the bulbous protuberances 40. The locking members 24, 26, and 30 can be formed from a substantially rigid material, such as a plastic or metal, and the legs 54 can be fixed or resiliently deflectable.

An intravenous tube 100 with an outer diameter within a given range greater than the distance between the narrowest distance between the legs 54, such as might be established by the bulbous portions 40, can be pressed between the bulbous portions 40 and into the retention volume 56. With that, the inwardly disposed portions 40 of the opposed legs 54 of each of the locking members 24, 26, and 30 and perhaps the opposed legs 54 themselves if the distance between them is less than the diameter of the tube 100 will tend to retain the intravenous tube 100 against inadvertent displacement from the retention volume 56 and the arcuate channel structure 16 in general.

It is again possible that the opposed legs 54 might not have inwardly disposed protuberances 40 but instead could merely have distances between at least portions of the legs 54 less than the outer diameter of the anticipated tube 100. The opposed legs 54 could be smooth, textured, or with gripping formations therealong such as teeth, ridges, angled fins, barbs, or any other gripping formation. The legs 54 could be substantially parallel, or they could be angled, such as by being angled toward one another whereby the distance between them will narrow toward their ends. The distance between the opposed legs 54 at their narrowest portions, whether at the inwardly disposed portions 40, any gripping formations, the tips of the legs 54, or even the overall legs 54 themselves, can be narrower than the outer diameter of the anticipated tube 100 and narrower than the distance between the first and second opposed sidewalls 50 of the arcuate channel structure 16. With that, the legs 54 of the locking members 24, 26, and 30 will exert a locking force on the tube 100.

With it again being noted that fewer or more locking members 24, 26, 28, and 30 could be provided, the locking members 24, 26, and 30 in the depicted embodiment are disposed with a locking member 24 disposed at a distal end of the first leg of the U-shaped arcuate channel structure 16 and a locking member 30 disposed at a distal end of the second leg of the arcuate channel structure 16. A central locking member 26 is disposed at a mid-portion of the arcuate channel structure 16, such as at what could be considered the base of the U-shape. The central locking member 26 could be unitary, segmented, or otherwise formed.

With the locking members 24, 26, and 30 so disposed, a segment of an intravenous tube 100 can be securely retained as in FIG. 7, such as by being snapped into place to be retained by the locking members 24, 26, and 30, to undergo a generally U-shaped directional change with the locking members 24 and 30 retaining first and second ends of the segment of the tube 100, central locking member 26 retaining mid-portions of the segment of the tube 100, and the portions of the tube 100 between the locking members 24, 26, and 30 being retained in the U-shaped channel 18 of the arcuate channel structure 16.

In this embodiment, the arcuate structure formed by the arcuate channel structure 16 and the locking members 24, 26, and 30 can be stabilized in a given configuration by a base panel 14, and the dedicated backing or cross member 20 can be foregone. The base panel 14 could again be a foam panel 36, or it could be a plastic or metal panel, or even some combination of layered or mixed materials. Adhesive coatings could be provided on either or both faces of the base panel 14. The arcuate channel structure 16 can be fixedly or selectively coupled to the base panel 14.

The legs of the arcuate channel structure 16 could again be adjustably retained, in this case by the base panel 14. The relative orientations of the legs of the arcuate channel structure 16 can be adjustable, and the angle of directional change of a retained intravenous tube 100 can be adjustable within a given range. For instance, where the legs of the arcuate channel structure 16 are coupled at or adjacent to the outboard edges of the base panel 14, the legs of the arcuate channel structure 16 will project somewhat away from one another and the angle of directional change of the tube 100 may be at or near a minimum, such as some angle less than 180 degrees. However, where the legs of the arcuate channel structure 16 are coupled toward a mid-portion of the base panel 14, the legs of the arcuate channel structure 16 will project somewhat toward one another and the angle of directional change of the tube 100 will be greater, such as at some angle in excess of 180 degrees. Of course, the range of adjustability of the angle of directional change will depend on the lateral dimension of the base panel 14. The adjustability of the angular configuration of the arcuate channel structure 16 can again be facilitated by a flexibility in the arcuate channel structure 16, in the coupling of the arcuate channel structure 16 with the locking members 24, 26, 28, and 30, or by some combination thereof or some other method.

It will again be noted that the flexibility of the arcuate channel structure 16 also permits it to conform to the contour of a patient's body and to permit movement of the patient while avoiding discomfort and dislodging of the arcuate guide for intravenous tubes 10. By a flexibility of, among other things, the arcuate channel structure 16, lateral movement of the legs of the arcuate channel structure 16 can be permitted as can bending and pivoting along and between the legs, including about a longitudinal axis.

The legs of the arcuate channel structure 16 could be retained relative to the base panel 14 in any effective manner, including adhesive, mechanical engagement, fasteners, or any other fastening or retention mechanism. The base panel 14, which is shaped and sized to correspond to the peripheral shape and size of the arcuate channel structure 16, has a plurality of apertures 46 spaced thereover. Where the legs of the arcuate channel structure 16 have a tooth 38 or opposed teeth 38 as in FIG. 4, the legs can be selectively engaged with an aperture 46 or apertures 46, such as in a snap-fit engagement. Alternatively, simple adhesive or another fastening method could be employed. In any case, the arcuate channel structure 16 and the base panel 14 can be readily and selectively engaged, and the angle of directional change provided by the arcuate guide for intravenous tubes 10 can be selectively modified. The arcuate retention structure 12, such as might be formed with the coupling of the arcuate channel structure 16 and the base panel 14, can be retained relative to a patient's body by any effective mechanism, including adhesive, tape, or any other method or combination thereof.

The base panel 14 can again have a window 34 disposed in a central portion thereof. The window 34 could be a translucent or transparent portion, or it may be entirely open, as by a cutout portion. Here, the window 34 is generally rectangular, but other shapes are possible. With such a window 34 provided, a visual inspection of the localized area of skin of the patient is again permitted.

The arcuate guide for intravenous tubes 10 of FIGS. 6 and 7 can be applied to a patient's body 200 to guide an intravenous tube 100 smoothly and in an arcuate manner of over an angle of directional change. The steps and the order of steps can vary according to the invention. One might adjust the arcuate channel structure 16 to cause there to be a given angle between the legs of the structure 16. The base panel 14 and the arcuate channel structure 16 can be coupled to secure the arcuate channel structure 16 at the desired angle. The joined arcuate channel structure 16 and the base panel 14 can be secured to a patient's body 200, such as the patient's arm. A portion of skin 204 aligned with the window 34 can be perceived. An intravenous tube 100 can be snapped into place within the locking members 24, 26, and 30 to be retained additionally by the arcuate channel structure 16 to undergo a given angle of directional change over a smooth, arcuate curvature. The intravenous needle 102 at the distal end of the intravenous tube 100 can be inserted into an injection site 202 on the patient's body 200. With this, liquid medicines and hydrative and nutritive liquids can be provided to the patient with the intravenous tube 100 smoothly changing direction, such as between the liquid supply vessel and the patient's arm 200, with reduced risks of kinking, occlusion, discomfort, and unintended withdrawal.

With certain details and embodiments of the present invention for an arcuate guide for intravenous tubes 10 disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the inventor. Those claims shall be deemed to include equivalent constructions insofar as they do not depart from the spirit and scope of the invention. It must be further noted that a plurality of the following claims may express certain elements as means for performing a specific function, at times without the recital of structure or material. As the law demands, any such claims shall be construed to cover not only the corresponding structure and material expressly described in this specification but also all equivalents thereof.

1. An arcuate guide for retaining and guiding an intravenous tube over an angular change of direction, the arcuate guide comprising:
a retention structure comprising an arcuate channel structure and a plurality of locking members disposed along the arcuate channel structure wherein the arcuate channel structure is generally U-shaped with first and second legs;
wherein the arcuate channel structure has an arcuate channel therein for receiving the intravenous tube and guiding the intravenous tube over an angular change of direction wherein the arcuate channel is defined by opposed sidewalls and a base portion;
wherein the plurality of locking members are retained in substantial alignment with the arcuate channel in the arcuate channel structure wherein the locking members have opposed sidewalls and a base portion that cooperate to define a retention volume;
a hinge portion disposed along the arcuate channel structure;
wherein there are at least first and second central locking members disposed at a mid-portion of the arcuate channel structure and wherein the hinge portion is disposed between the first and second central locking members disposed at the mid-portion of the arcuate channel structure.

2. The arcuate guide of claim 1 wherein the arcuate channel in the arcuate channel structure has a substantially U-shaped cross section.

3. (canceled)

4. The arcuate guide of claim 2 wherein the arcuate channel in the arcuate channel structure has radiused distal edges.

5. The arcuate guide of claim 1 wherein the first and second legs of the arcuate channel structure are selectively adjustable in relative angular configuration such that the angular change of direction of the intravenous tube can be selectively adjusted over a range of angular change of direction of the intravenous tube.

6-8. (canceled)

9. An arcuate guide for retaining and guiding an intravenous tube over an angular change of direction, the arcuate guide comprising:
a retention structure comprising an arcuate channel structure wherein the arcuate channel structure is generally U-shaped with first and second legs;
wherein the arcuate channel structure has an arcuate channel therein for receiving the intravenous tube and guiding the intravenous tube over an angular change of direction of the intravenous tube;
a support member wherein the first and second legs of the arcuate channel structure can be selectively retained by the support member at selected angles of orientation over a range of angular change of direction of the intravenous tube that encompasses from less than 180 degrees to greater than 180 degrees.

10. The arcuate guide of claim 9 wherein the support member comprises a cross member.

11. The arcuate guide of claim 9 wherein the support member comprises a base panel.

12. An arcuate guide for retaining and guiding an intravenous tube over an angular change of direction, the arcuate guide comprising:
a retention structure comprising an arcuate channel structure wherein the arcuate channel structure is generally U-shaped with first and second legs;
wherein the arcuate channel structure has an arcuate channel therein for receiving the intravenous tube and guiding the intravenous tube over an angular change of direction;
a support member comprising a base panel for retaining the arcuate channel structure;
a plurality of apertures through the base panel of the support member and wherein the first and second legs can be selectively retained relative to the plurality of apertures at selected angles of orientation over a range of angles or orientation by at least one tooth that projects from the first leg for being received through a first aperture of the plurality of apertures and at least one tooth that projects from the second leg for being received through a second aperture of the plurality of apertures.

13-14. (canceled)

15. The arcuate guide of claim 5 wherein the range of angular change of direction of the intravenous tube encompasses from less than 180 degrees to greater than 180 degrees.

16. The arcuate guide of claim 1 wherein the arcuate channel in the arcuate channel structure is defined by first and second opposed sidewalls separated by a given effective distance, wherein each locking member has opposed sidewalls separated by a given effective distance, and wherein the effective distance between the opposed sidewalls of the locking members is less than the effective distance between the first and second opposed sidewalls of the arcuate channel in the arcuate channel structure.

17. The arcuate guide of claim 16 wherein there is a locking member disposed adjacent to a first end of the arcuate channel in the arcuate channel structure and a locking member disposed adjacent to a second end of the arcuate channel in the arcuate channel structure.

18. The arcuate guide of claim 16 wherein the opposed sidewalls of the locking members are formed from substantially rigid material.

19. The arcuate guide of claim 18 wherein the opposed sidewalls of the locking member are resiliently deflectable.

20. The arcuate guide of claim 1 further comprising a support member for retaining the arcuate retention structure wherein the support member has a first face and a second face.

21. The arcuate guide of claim 20 further comprising adhesive disposed on at least the first face of the support member for selectively retaining the support member and the arcuate guide relative to a patient's skin.

22. The arcuate guide of claim 21 further comprising adhesive disposed on the second face of the support member for retaining the arcuate retention structure.

23. The arcuate guide of claim 20 wherein the support member comprises a panel of material for being interposed between the arcuate retention structure and a patient's skin.

24. An arcuate guide for retaining and guiding an intravenous tube over an angular change of direction, the arcuate guide comprising:
a retention structure comprising an arcuate channel structure wherein the arcuate channel structure is generally U-shaped with first and second legs whereby a central portion is defined between the first and second legs of the arcuate channel structure;
wherein the arcuate channel structure has an arcuate channel therein for receiving the intravenous tube and guiding the intravenous tube over an angular change of direction of the intravenous tube;
a support member comprising a base panel for retaining the arcuate channel structure;
a window in the support member for permitting a visual perception of the patient's skin wherein the window is disposed in the central portion of the base panel between the first and second legs of the arcuate channel structure.

25. The arcuate guide of claim 24 wherein the window comprises a translucent portion.

26. The arcuate guide of claim 1 wherein the hinge comprises a bi-directional hinge that permits lateral movement of the first and second legs of the arcuate channel structure and pivoting between the first and second legs.
